(19) United States
(12) Patent Application Publication
(10) Pub. No.: US 2017/0035764 A1
(43) Pub. Date: Feb. 9, 2017

(54) SEXUAL DYSFUNCTION THERAPEUTIC GEL

(71) Applicant: SYNERGISTIC THERAPEUTICS, LLC, NAPLES, FL (US)

(72) Inventors: ANTHONY H. SALCE, JR., NAPLES, FL (US); WILLIAM F. GREENWOOD, FAIRFIELD, CT (US); SHIVSANKAR MISIR, NAPLES, FL (US)

(73) Assignee: SYNERGISTIC THERAPEUTICS, LLC, NAPLES, FL (US)

(21) Appl. No.: 15/227,477

(22) Filed: Aug. 3, 2016

Related U.S. Application Data
(60) Provisional application No. 62/200,304, filed on Aug. 3, 2015.

(51) Int. Cl. A61K 31/519; A61K 45/06; A61K 9/16; A61J 3/02; A61K 9/00; A61K 9/06

(52) U.S. Cl. CPC A61K 31/519; A61K 9/0034; A61K 9/06; A61K 9/1694; A61J 3/02; A61K 45/06

(57) ABSTRACT

Technologies are described for formulations and methods to produce a topical formulation. Topical formulations may comprise a β-carboline compound. The β-carboline compound may be in particulate form. The particle size of the particulate form of the β-carboline compound may be in a range of from about 1 micron to about 40 microns in size.

### BACKGROUND

Cyclic guanosine monophosphate specific phosphodiesterase (cGMP-specific PDE) inhibitors, such as sildenafil, may produce biochemical, physiological and clinical effects. These effects may include modulation of smooth muscle, renal, hemostatic, inflammatory, and/or endocrine function. Type 5 cGMP-specific phosphodiesterase (PDE5) may be a cGMP hydrolyzing enzyme in vascular smooth muscle.

### SUMMARY

In some examples topical formulations are described. The topical formulations may comprise a β-carboline compound. The β-carboline compound may be in particulate form. The particle size of the particulate form of the β-carboline compound may be in a range of from about 1 micron to about 40 microns in size.

In some examples, methods to produce a topical formulation are described. The methods may comprise depositing a β-carboline compound into a chamber. The methods may comprise milling the β-carboline compound into a powder. The powder of the β-carboline compound may include the β-carboline compound in particulate form. The particle size of the particulate form of the β-carboline compound may be in a range of from about 1 micron to about 40 microns in size. The methods may comprise adding a solvent with the powder in the chamber. The methods may comprise mixing the solvent with the powder in the chamber to form a solution. The methods may comprise adding a base gel to the solution in the chamber. The methods may comprise mixing the base gel and the solution to form the topical formulation.

In some examples, topical formulations are described. The topical formulations may comprise a β-carboline compound in particulate form. The particle size of the particulate form of the β-carboline compound may be in a range of from about 1 micron to about 40 microns in size. The topical formulation may comprise a solvent. The topical formulation may comprise a base gel. The β-carboline compound may be in a range of about 1 to about 10 weight % of the topical formulation.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates an example system that can be utilized to produce a sexual dysfunction therapeutic gel;

FIG. 2 illustrates an example system that can be utilized to produce a sexual dysfunction therapeutic gel; and

FIG. 3 illustrates a flow diagram of an example process to produce a sexual dysfunction therapeutic gel;

all arranged according to at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

It will be understood that any compound, material or substance which is expressly or implicitly disclosed in the specification and/or recited in a claim as belonging to a group or structurally, compositionally and/or functionally related compounds, materials or substances, includes individual representatives of the group and all combinations thereof.

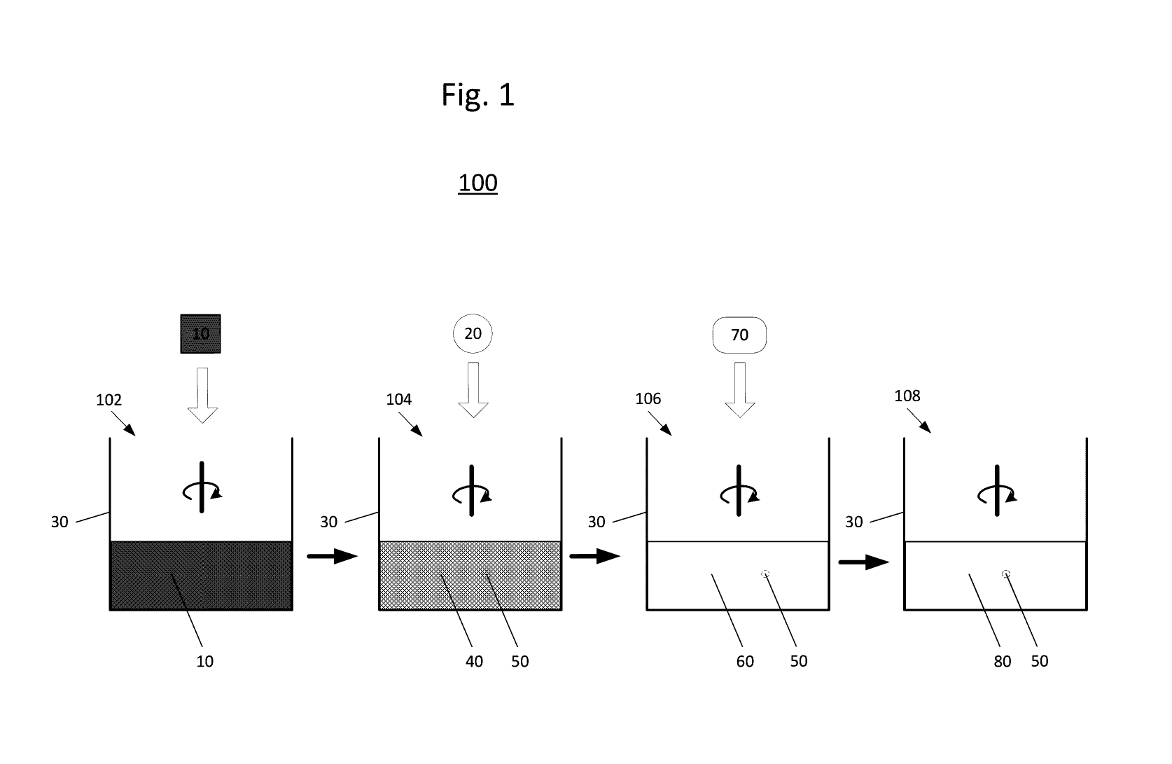

FIG. 1 illustrates an example system that can be utilized to produce a sexual dysfunction therapeutic gel, arranged in accordance with at least some embodiments presented herein. As discussed in more detail below, a sexual dysfunction therapeutic gel may be effective in the treatment of female sexual dysfunction.

System 100 may include a compound 10, a solvent 20, and a chamber 30. Compound 10 may be a β-carboline compound. Compound 10 may be a PDE5 inhibitor and may exhibit poor solubility. Compound 10 may include sildenafil and have a chemical formula C₂₂H₃₀N₆O₄S. As shown at 102, compound 10 may be deposited into chamber 30. Compound 10 in chamber 30 may be milled to form a fine powder 40 (as shown at 104). Powder 40 of compound 10 may include particulates 50 with a particle size from 1 micron to 40 microns. In another embodiment, particulates 50 may have a particle size from 1 micron to 30 microns. In another embodiment, particulates 50 may have at least 90% of particles from 1 micron to 25 microns in size. In another embodiment, particulates 50 may have at least 90% of the particles from 1 micron to 10 microns in size.

As shown at 104, solvent 20 may be added with powder 40, including particulates 50, in chamber 30. Solvent 20 may be mixed with powder 40, including particulates 50, until a clear solution 60 is formed (as shown at 106). Solution 60 may include powder 40, including particulates 50, dispersed in solvent 20. Mixing may be performed either by hand or machine. Solvent 20 may include propylene glycol, water, alcohol, or mineral oil and combinations thereof.

At 106, a base gel 70 may be added to chamber 30 with solution 60. Base gel 70 may be mixed with solution 60 to produce sexual dysfunction therapeutic gel 80 (as shown at 108). Mixing may be performed either by hand or machine. An amount of base gel 70 may be added in proportion to an amount of solution 60 so as to achieve a desired weight percentage of β-carboline compound 10 in therapeutic gel 80. Base gel 70 may include a moisturizing skin gel. Base gel 70 may include VANICREAM. Base gel 70 may substantially allow absorption of base gel 70 through the passageways of skin. Base gel 70 in therapeutic gel 80 may substantially prevent fluid washout of solution 60 from therapeutic gel 80 and may provide stability to sexual dysfunction therapeutic gel 80.

Base gel 70 may be added to solution 60 so that compound 10 comprises from 0.5% by weight to 50.0% by weight of sexual dysfunction therapeutic gel 80. Base gel 70 may be added to solution 60 so that compound 10, comprises about 1 to about 10 weight % of sexual dysfunction therapeutic gel 80. A of size of particulates 50 in sexual dysfunction therapeutic gel 80 may provide for a formulation of stable pharmaceutical compositions. A size of particulates 50 in sexual dysfunction therapeutic gel 80 may provide a desired bioavailability of particulates 50 of sexual dysfunction therapeutic gel 80. Sexual dysfunction therapeutic gel 80 may be topically applied to the clitoris of the vagina and may provide a rapid therapeutic effect. Sexual dysfunction therapeutic gel 80, including particulates 50 of compound 10, may be topically applied to the clitoris of the vagina and be effective in the treatment of female sexual dysfunction.

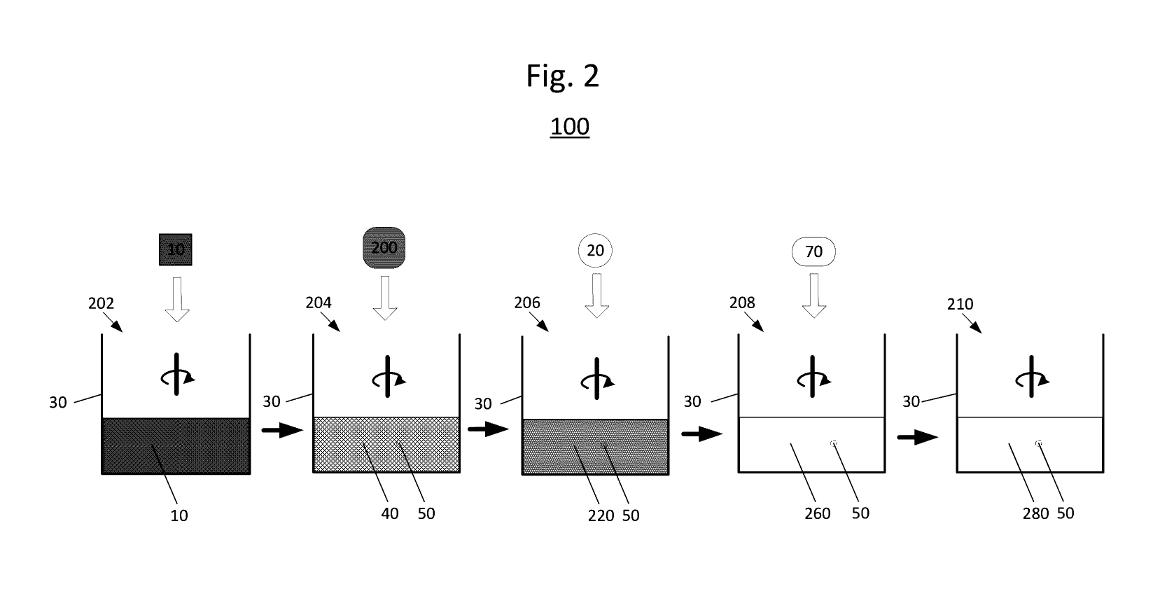

FIG. 2 illustrates an example system that can be utilized to produce a sexual dysfunction therapeutic gel, arranged in accordance with at least some embodiments presented herein. Those components in FIG. 2 that are labelled identically to components of FIG. 1 may not be described again for the purposes of clarity and brevity.

At 202 compound 10 may be added to chamber 30. Compound 10 may be milled into fine powder 40, with particulates 50, in chamber 30 (as shown at 204). At 204, compound 200 may be added to chamber 30. Compound 200 in chamber 30 may be milled into a fine powder and mixed with powder 40 to form powder 220 (as shown at 206). Powder 220 of compound 10 and compound 200 may include particulates 50 of compound 10.

Compound 200 may be a calcium channel blocker. Compound 200 may be verapamil and may have a formula of C₂₇F₃₈N₂O₄. Compound 200 may be a phenylalkylamine calcium channel blocker and may treat hypertension and cluster headaches. Compound 200 may be an ionic calcium influx inhibitor more commonly known as a calcium channel blocking agent. Compound 200 may inhibit the transmembrane influx of extracellular calcium ions across the membrane of myocardial cells and vascular smooth muscle cells. Compound 200, by inhibiting calcium influx, may inhibit the contractile processes, and may thereby dilate the main outer layers of the skin to allow penetration of the therapeutic gel.

At 206, solvent 20 may be added with powder 220, including particulates 50, in chamber 30. Solvent 20 may be mixed with powder 220, including particulates 50, until a clear solution 260 is formed (as shown at 208). Solution 260 may include powder 220, including particulates 50, dispersed in solvent 20. Mixing may be performed either by hand or machine. Solvent 20 may include propylene glycol, water, alcohol, or mineral oil and combinations thereof.

At 208, base gel 70 may be added to chamber 30 with solution 260. Base gel 70 may be mixed with solution 260 to produce sexual dysfunction therapeutic gel 280 (as shown at 210). Mixing may be performed either by hand or machine. An amount of base gel 70 may be added in proportion to an amount of solution 260 so as to achieve a desired weight percentage of β-carboline compound 10 in therapeutic gel 280.

Base gel 70 may be added to solution 260 so that compound 10 may comprise from 0.5% by weight to 50.0% by weight of sexual dysfunction therapeutic gel 280. Base gel 70 may be added to solution 260 so that compound 10, may comprise about 1 to about 10 weight % of sexual dysfunction therapeutic gel 280. Base gel 70 may be added to solution 260 so that compound 260, may comprise about 0.001 to about 0.005 weight % of sexual dysfunction therapeutic gel 280.

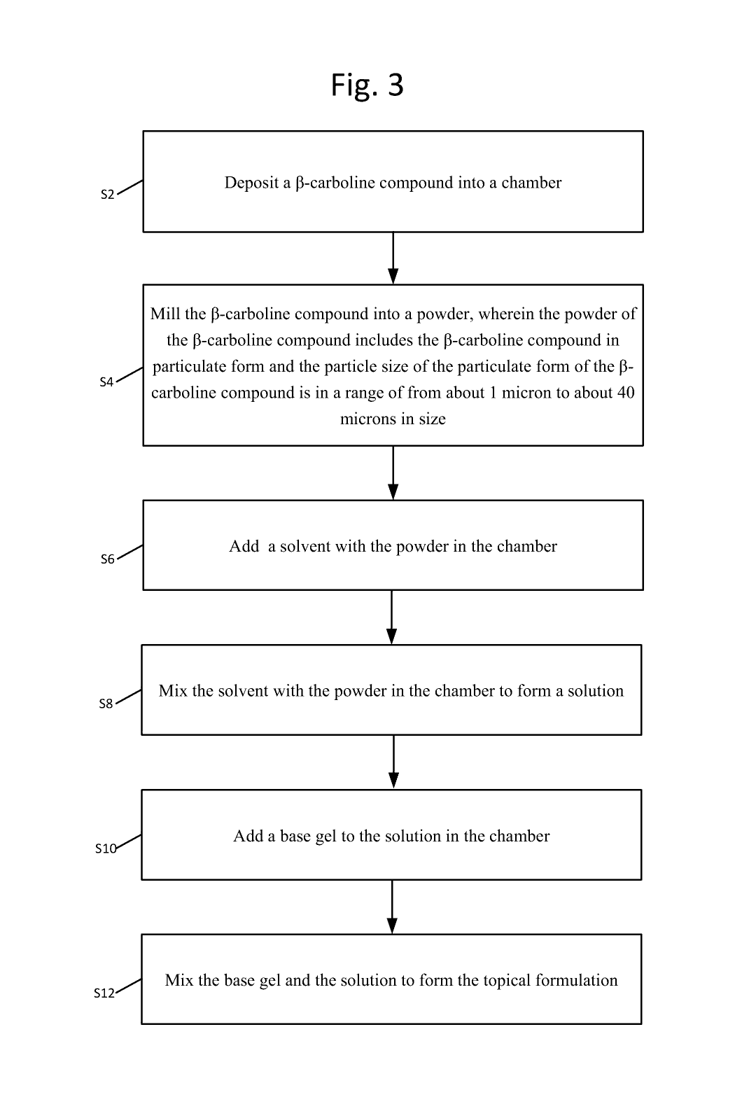

FIG. 3 illustrates a flow diagram of an example process to produce a sexual dysfunction therapeutic gel 80. The process in FIG. 3 could be implemented using, for example, system 100 discussed above. An example process may include one or more operations, actions, or functions as illustrated by one or more of blocks S2, S4, S6, S8, and/or S10. Although illustrated as discrete blocks, various blocks may be divided into additional blocks, combined into fewer blocks, or eliminated, depending on the desired implementation.

Processing may begin at block S2, “Deposit a β-carboline compound into a chamber.” At block S2, a β-carboline compound may be deposited into a chamber. The β-carboline compound may be a PDE5 inhibitor and may exhibit poor solubility The β-carboline compound may include sildenafil and have a chemical formula C₂₂H₃₀N₆O₄S.

Processing may continue from block S2 to block S4, “Mill the β-carboline compound into a powder, wherein the powder of the β-carboline compound includes the β-carboline compound in particulate form and the particle size of the particulate form of the β-carboline compound is in a range of from about 1 micron to about 40 microns in size.” At block S4, the β-carboline compound may be milled into a powder. The powder of the β-carboline compound may include the β-carboline compound in particulate form. The particle size of the particulate form of the β-carboline compound may be in a range of from about 1 micron to about 40 microns in size.

Processing may continue from block S4 to block S6, “Add a solvent with the powder in the chamber.” At block S6, a solvent may be added with the powder in the chamber. The solvent may include propylene glycol, water, alcohol, or mineral oil and combinations thereof.

Processing may continue from block S6 to block S8, “Mix the solvent with the powder in the chamber to form a solution.” At block S8, the solvent may be mixed with the powder in the chamber. The solvent may be added and mixed with the powder in the chamber until a clear solution is formed. The clear solution may include particulates of the powder dispersed in the solvent. Mixing may be performed either by hand or machine.

Processing may continue from block S8 to block S10, “Add a base gel to the solution in the chamber.” At block S10, a base gel may be added to the solution in the chamber. An amount of the base gel may be added in proportion to an amount of the solution in the chamber so as to achieve a desired weight percentage of the β-carboline compound in the formulation. The base gel may include a moisturizing skin gel. The base gel may include VANICREAM. The base gel may substantially allow absorption of the base gel through the passageways of skin. The base gel may substantially prevent fluid washout of the solution in the formulation. The base gel may provide stability to the formulation.

Processing may continue from block S10 to block S12, “Mix the base gel and the solution to form the topical formulation.” At block S12, the base gel may be mixed with the solution to form the topical formulation.

A system in accordance with the present disclosure may be effective for the treatment of sexual dysfunction, such as female sexual arousal disorder. An embodiment of the present application may provide a more rapid achievement of maximum blood concentration of β-carboline-containing pharmaceutical compositions than current treatments such as orally delivered methods. A system in accordance with the present disclosure may be effective in less time than current treatments such as orally delivered methods.

In some cases, women who object in principle to the idea of taking a pill to enhance sexual functioning may have no difficulty accepting a topical gel. In an example, an embodiment of the present application may half the dose of an oral preparation. A β-carboline compound may enhance all four phases of a sexual response cycle: desire, excitement, orgasm, and resolution. A β-carboline compound may be helpful in the treatment of a variety of female sexual problems, including hypoactive sexual desire disorder, female sexual arousal disorder, female orgasmic disorder, inadequate lubrication, dyspareunia, and sexual dysfunction secondary to medication or illness.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A topical formulation comprising a β-carboline compound in particulate form wherein the particle size of the particulate form of the β-carboline compound is in a range of from about 1 micron to about 40 microns in size.

2. The topical formulation of claim 1, wherein the β-carboline compound includes sildenafil and has a formula of C₂₂H₃₀N₆O₄S.

3. The topical formulation of claim 1, wherein the particle size of the particulate form of the β-carboline compound is in a range of from about 1 micron to about 30 microns in size.

4. The topical formulation of claim 1, wherein the particle size of at least 90% of the particulate form of the β-carboline compound is in a range of from about 1 micron to about 25 microns in size.

5. The topical formulation of claim 1, wherein the particle size of at least 90% of the particulate form of the β-carboline compound is in a range of from about 1 micron to about 10 microns in size.

6. The topical formulation of claim 1, further comprising a solvent.

7. The topical formulation of claim 7, wherein the solvent includes propylene glycol, water, alcohol, or mineral oil and combinations thereof.

8. The topical formulation of claim 1, wherein the β-carboline compound is in a range of about 0.5 to about 50.0 weight % of the topical formulation.

9. The topical formulation of claim 1, wherein the β-carboline compound is in a range of about 1 to about 10 weight % of the topical formulation.

10. The topical formulation of claim 1, further comprising verapamil and the verapamil has a formula of C₂₇H₃₈N₂O₄ and is in a range of about 0.001 to about 0.005 weight % of the topical formulation.

11. A method to produce a topical formulation, the method comprising:
depositing a β-carboline compound into a chamber;
milling the β-carboline compound into a powder, wherein the powder of the β-carboline compound includes the β-carboline compound in particulate form and the particle size of the particulate form of the β-carboline compound is in a range of from about 1 micron to about 40 microns in size;
adding a solvent with the powder in the chamber;
mixing the solvent with the powder in the chamber to form a solution;
adding a base gel to the solution in the chamber; and
mixing the base gel and the solution to form the topical formulation.

12. The method of claim 11, wherein the β-carboline compound is sildenafil and has a formula of C₂₂H₃₀N₆O₄S.

13. The method of claim 11, wherein the particle size of the particulate form of the β-carboline compound is in a range of from about 1 micron to about 30 microns in size.

14. The method of claim 11, wherein the particle size of at least 90% of the particulate form of the β-carboline compound is in a range of from about 1 micron to about 25 microns in size.

15. The method of claim 11, wherein the particle size of at least 90% of the particulate form of the β-carboline compound is in a range of from about 1 micron to about 10 microns in size.

16. The method of claim 11, wherein the solvent includes propylene glycol, water, alcohol, or mineral oil and combinations thereof.

17. The method of claim 11, wherein the β-carboline compound is in a range of about 1 to about 10 weight % of the topical formulation.

18. The method of claim 11, wherein the powder is a first powder, the method further comprising, prior to adding the solvent to the chamber:
adding a calcium channel blocker to the chamber;
milling the calcium blocker into a second powder; and
mixing the first powder and the second powder to form a third powder, wherein the third powder includes the β-carboline compound in particulate form.

19. A topical formulation comprising:
a β-carboline compound in particulate form wherein the particle size of the particulate form of the β-carboline compound is in a range of from about 1 micron to about 40 microns in size;
a solvent; and
a base gel, wherein the β-carboline compound is in a range of about 1 to about 10 weight % of the topical formulation.

20. The therapeutic formulation of claim 19 wherein the particle size of at least 90% of the particulate form of the β-carboline compound is in a range of from about 1 micron to about 10 microns in size.
